(19) United States
(12) Patent Application Publication
(10) Pub. No.: US 2026/0216127 A1
(43) Pub. Date: Jul. 30, 2026

(54) COMBINATIONS OF A UROTHILIN AND A KETONE BODY

(71) Applicant: Amazentis SA, Lausanne (CH)

(72) Inventors: Christopher L. Rinsch, Lausanne (CH); Anurag Singh, Lausanne (CH); Davide D'Amico, Renens (CH)

(21) Appl. No.: 19/141,811

(22) PCT Filed: Dec. 19, 2023
(86) PCT No.: PCT/EP23/86774

§ 371 Date: Jun. 20, 2025

(30) Foreign Application Priority Data
Dec. 20, 2022 (GB) 2219317.1

(51) Int. Cl. A61K 31/37; A61K 31/121; A61K 31/191; A61P 29/00

(52) U.S. Cl. CPC A61K 31/37; A61K 31/121; A61K 31/191; A61P 29/00

(57) ABSTRACT

The invention relates to methods involving administration of a combination of a urolithin and one or more ketone body, resulting in the provision of beneficial health effects, for example improved resistance to inflammation, mitochondrial function and cellular metabolism. The methods are useful, e.g. for improving the health and wellbeing of subjects, particularly the elderly or frail; and for improving fitness, muscle performance and/or endurance of those engaging in exercise. The methods are also useful in treating or preventing various conditions, e.g. conditions associated with inadequate mitochondrial activity, excessive inflammation, and/or muscle-related disorders. The invention further relates to compositions comprising a urolithin and one or more ketone body

### FIELD

The invention relates to methods involving administration of a combination of a urolithin and one or more ketone body, resulting in the provision of beneficial health effects, for example improved resistance to inflammation, mitochondrial function and cellular metabolism. The methods are useful, e.g. for improving the health and wellbeing of subjects, particularly the elderly or frail; and for improving fitness, muscle performance and/or endurance of those engaging in exercise. The methods are also useful in treating or preventing various conditions, e.g. conditions associated with inadequate mitochondrial activity, excessive inflammation, and/or muscle-related disorders. The invention further relates to compositions comprising a urolithin and one or more ketone body.

### BACKGROUND

Urolithins have been proposed as treatments of a variety of conditions related to inadequate mitochondrial activity, including obesity, reduced metabolic rate, metabolic syndrome, diabetes mellitus, cardiovascular disease, hyperlipidemia, neurodegenerative diseases, cognitive disorders, mood disorders, stress, and anxiety disorders; for weight management, or to increase muscle performance or mental performance. See WO2012/088519 (Amazentis SA). In WO2007/127263 (The Regents of the University of California), the use of urolithins for the treatment of various neoplastic diseases is described.

International patent publication WO2014/004902 (derived from application PCT/US2013/48310) discloses a method of increasing autophagy, including specifically mitophagy, in a cell, comprising contacting a cell with an effective amount of a urolithin or a pharmaceutically acceptable salt thereof, thereby increasing autophagy, including specifically mitophagy, in the cell. Administration may be to a subject having a disease or condition selected from metabolic stress, cardiovascular disease, endothelial cell dysfunction, sarcopenia, muscle degenerative disease, Duchenne muscular dystrophy, alcoholic liver disease, non-alcoholic fatty liver disease, drug-induced liver or muscle injury, α1-antitrypsin deficiency, ischemia/reperfusion injury, inflammation, aging of the skin, inflammatory bowel disease, Crohn's disease, obesity, metabolic syndrome, type II diabetes mellitus, hyperlipidemia, osteoarthritis, neurodegenerative disease, Alzheimer's disease, Huntington's disease, Parkinson's disease, amyotrophic lateral sclerosis, age-related macular degeneration, mitochondrial diseases (including for example poor growth, loss of muscle coordination, muscle weakness, visual problems, hearing problems, heart disease, liver disease, kidney disease, gastrointestinal disorders, respiratory disorders, neurological problems, autonomic dysfunction sometimes learning disabilities, and dementia as a result of mitochondrial disease), muscle diseases; cancer, cognitive disorder, stress, and mood disorder.

In particular, urolithins have been proposed as treatments for muscle-related pathological conditions. Muscle-related pathological conditions include myopathies and neuromuscular diseases. Examples of such conditions include Duchenne muscular dystrophy, acute sarcopenia, for example muscle atrophy and/or cachexia, for example associated with burns, bed rest, limb immobilization, or major thoracic, abdominal, neck and/or orthopedic surgery, amyotrophic lateral sclerosis and multiple sclerosis. Age-related muscle-loss is an especially prevalent condition. Cachexia due to prolonged immobilization or other diseases, for example cancer, are other conditions that are often characterised by poor muscle performance.

Another biological function of Urolithin A is the attenuation of detrimental inflammatory responses. This is particularly clinically relevant because aging and most age-related diseases are associated with chronic, low-grade inflammation. This process, recently named inflammaging, contributes to age-related decline in cellular and organismal function.

Effective muscle function and physical performance is important for having a high quality of life at all ages in healthy individuals as well as in those individuals suffering from a disease, especially the elderly. Improved muscle performance is of particular interest to athletes. For example an increase in muscular contraction strength, increase in amplitude of muscle contraction, or shortening of muscle reaction time between stimulation and contraction are all of benefit to individuals, especially athletes. For elderly suffering from age related decline in muscle function including muscle loss/wasting or individuals suffering from cachexia muscle wasting, an improvement in muscle and physical performance is important for basic aspects of daily functioning such as walking speed and distance they can walk unassisted.

Ketone bodies are a group of carbonyl-containing compounds, including acetoacetate, 3-hydroxybutyrate (BHB) and acetone, which are produced by the liver from fatty acids under certain conditions. They are known to induce mitochondrial biogenesis, and have been suggested as having benefits on muscle performance, metabolism, neuroprotection, healthy aging, and cardiovascular health, cognitive performance, and mental health. It has also been suggested that ketone bodies are useful in the treatment of cancer.

### SUMMARY

The invention provides a combination comprising or consisting of a urolithin (or a salt, prodrug, metabolite or derivative thereof) and a ketone body (or a salt, prodrug, metabolite or derivative thereof).

The invention provides a method for providing beneficial health effects to a subject, the method comprising administering to the subject a combination comprising or consisting of a urolithin (or a salt, prodrug, metabolite or derivative thereof) and a ketone body (or a salt, prodrug, metabolite or derivative thereof).

The invention provides a combination comprising or consisting of a urolithin (or a salt, prodrug, metabolite or derivative thereof) and a ketone body (or a salt, prodrug, metabolite or derivative thereof) for the provision of beneficial health effects to a subject.

The invention provides use of a combination comprising or consisting of a urolithin (or a salt, prodrug, metabolite or derivative thereof) and a ketone body (or a salt, prodrug, metabolite or derivative thereof) to provide beneficial health effects to a subject.

The invention provides use of a combination comprising or consisting of a urolithin (or a salt, prodrug, metabolite or derivative thereof) and a ketone body (or a salt, prodrug, metabolite or derivative thereof) in the manufacture of a medicament which provides beneficial health effects to a subject.

The invention provides a method of therapy comprising administration of a combination comprising or consisting of a urolithin (or a salt, prodrug, metabolite or derivative thereof) and a ketone body (or a salt, prodrug, metabolite or derivative thereof).

The invention provides a combination comprising or consisting of a urolithin (or a salt, prodrug, metabolite or derivative thereof) and a ketone body (or a salt, prodrug, metabolite or derivative thereof) for use as a medicament.

The invention provides use of a combination comprising or consisting of a urolithin (or a salt, prodrug, metabolite or derivative thereof) and a ketone body (or a salt, prodrug, metabolite or derivative thereof) in the manufacture of a medicament.

The invention provides a composition comprising a urolithin (or a salt, prodrug, metabolite or derivative thereof) and a ketone body (or a salt, prodrug, metabolite or derivative thereof).

The invention provides a method for providing beneficial health effects to a subject, the method comprising administering to the subject a composition comprising a urolithin (or a salt, prodrug, metabolite or derivative thereof) and a ketone body (or a salt, prodrug, metabolite or derivative thereof).

The invention provides a composition comprising a urolithin (or a salt, prodrug, metabolite or derivative thereof) and a ketone body (or a salt, prodrug, metabolite or derivative thereof) for the provision of beneficial health effects to a subject.

The invention provides use of a composition comprising a urolithin (or a salt, prodrug, metabolite or derivative thereof) and a ketone body (or a salt, prodrug, metabolite or derivative thereof) to provide beneficial health effects to a subject.

The invention provides use of a composition comprising a urolithin (or a salt, prodrug, metabolite or derivative thereof) and a ketone body (or a salt, prodrug, metabolite or derivative thereof) in the manufacture of a medicament which provides beneficial health effects to a subject.

The invention provides a method of therapy comprising administration of a composition comprising a urolithin (or a salt, prodrug, metabolite or derivative thereof) and a ketone body (or a salt, prodrug, metabolite or derivative thereof).

The invention provides a composition comprising a urolithin (or a salt, prodrug, metabolite or derivative thereof) and a ketone body (or a salt, prodrug, metabolite or derivative thereof) for use as a medicament.

The invention provides use of a composition comprising a urolithin (or a salt, prodrug, metabolite or derivative thereof) and a ketone body (or a salt, prodrug, metabolite or derivative thereof) in the manufacture of a medicament.

The invention provides a kit comprising a composition comprising a ketone body (or a salt, prodrug, metabolite or derivative thereof) together with a separate composition comprising a urolithin (or a salt, prodrug, metabolite or derivative thereof)

### DETAILED DESCRIPTION

The invention provides a combination comprising or consisting of a urolithin (or a salt, prodrug, metabolite or derivative thereof) and a ketone body (or a salt, prodrug, metabolite or derivative thereof).

### Ketone Body

A ketone body is one of a group of carbonyl-containing compounds which are produced by the liver from fatty acids under certain conditions, including acetoacetate, β-hydroxybutyrate and acetone. Ketone bodies are produced when fatty acid levels rise in the body and are metabolised by the body for energy.

Preferably, the ketone body for use in the invention is selected from acetoacetate or a salt, prodrug, metabolite or derivative thereof, β-hydroxybutyrate or a salt, prodrug, metabolite or derivative thereof (e.g. (R)-β-hydroxybutyrate or a salt, prodrug, metabolite or derivative thereof or (S)-β-hydroxybutyrate or a salt, prodrug, metabolite or derivative thereof), and acetone or a salt, prodrug, metabolite or derivative thereof, or a mixture thereof. Ketone bodies are selected from β-ketopentanoate and β-hydroxypentanoate or salts, prodrugs, metabolites or derivatives thereof.

In some combinations and compositions of the invention, the ketone body is acetoacetate or a salt, prodrug, metabolite or derivative thereof, or a mixture thereof. In some embodiments of the invention, the ketone body is β-hydroxybutyrate or a salt, prodrug, metabolite or derivative thereof, or a mixture thereof. In some embodiments of the invention, the ketone body is acetone or a salt, prodrug, metabolite or derivative thereof, or a mixture thereof.

In some combinations and compositions of the invention, the ketone body is acetoacetate. In some combinations and compositions of the invention, the ketone body is β-hydroxybutyrate. In some combinations and compositions of the invention, the ketone body is acetone.

In some embodiments of the invention, the combination or composition may include more than one ketone body (or salt, prodrug, metabolite or derivative thereof). In some embodiments of the invention, the combination and composition comprises acetoacetate and β-hydroxybutyrate. In some embodiments of the invention, combination and composition comprises acetoacetate and acetone. In some embodiments of the invention, the combination and composition comprises β-hydroxybutyrate and acetone. In some embodiments of the invention, the combination and composition comprises acetoacetate, β-hydroxybutyrate and acetone.

The present invention also encompasses use of suitable salts of ketone bodies, e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

The present invention also encompasses use of prodrugs and derivatives of ketone bodies. A ketone body derivative or a ketone body prodrug is a compound or species which may be converted or metabolised to a ketone body. Suitable ketone body derivatives and ketone body prodrugs include, for example, ketone body esters (e.g. [(3R)-3-hydroxybutyl](3S)-3-hydroxybutanoate, [(3S)-3-hydroxybutyl](3R)-3-hydroxybutanoate, [(3S)-3-hydroxybutyl](3S)-3-hydroxybutanoate, or esters formed using butanediol, especially, butane-1,3-diol, altrose, arabinose, dextrose, erythrose, fructose, galactose, glucose, glycerol, gulose, idose, lactose, lyxose, mannose, ribitol, ribose, ribulose, sucrose, talose, threose, xylitol, xylose, galactosamine, glucosamine, mannosamine, N-acetylglucosamine, mannitol, sorbitol, threitol, and (S)-1,2-propanediol), oligomers of the ketone bodies or esters thereof, (R)-1,3-butandiol, triacetin, free fatty acids and triglycerides. Preferred ketone body derivatives and ketone body prodrugs are β-hydroxybutyrate-(R)-1,3-butanediolmonoester.

The present invention also encompasses use of metabolites of ketone bodies.

Those skilled in the art of organic chemistry will appreciate that many organic compounds can form complexes with solvents in which they are reacted or from which they are precipitated or crystallized. These complexes are known as “solvates”. It will be understood by the skilled person that the invention also encompasses solvates of the ketone bodies, as well as solvates of salts, prodrugs, metabolites and derivatives thereof. Solvates include those where the associated solvent is pharmaceutically acceptable. A hydrate (in which the associated solvent is water) is an example of a solvate.

### Urolithins:

Urolithins are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative formula (I) shown below. The structures of some particularly common urolithins are described in the table below, with reference to formula (I).

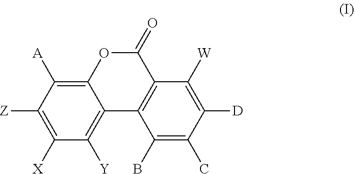

wherein A, B, C, D, W, X, Y and Z are each independently selected from H and OH.

| | Substituent of structure (I) | | | | | |
|---|---|---|---|---|---|---|
| | A | B | C | D | W, X and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO2014/004902 and WO2019/0168972.

Particularly suitable compounds for use in the invention are the naturally occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is Urolithin A. Preferably, the Urolithin used in a formulation of the invention is Urolithin A, Urolithin B, Urolithin C and/or Urolithin D. More preferably, the urolithin used in a formulation of the invention is Urolithin A or Urolithin B. Most preferably, the Urolithin used in a formulation of the invention is Urolithin A.

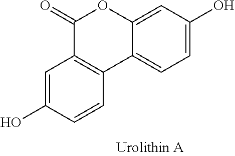 [SMILES: O=c1oc2cc(O)ccc2c2ccc(O)cc12]

Isourolithin A and B may also be mentioned. When W, X and Y are all H, and A, B and D are all H, and C and Z are both OH, then the compound is Iso-urolithin A. When W, X, Y and Z are all H, and A, B and D are all H, and C is OH, then the compound is Iso-urolithin B.

The present invention also encompasses use of suitable salts of urolithins, e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

The present invention also encompasses use of prodrugs of urolithins, for example amino acid derivatives of urolithins. One preferred group of prodrugs of urolithins for use in the present invention are the compounds of formula (II)

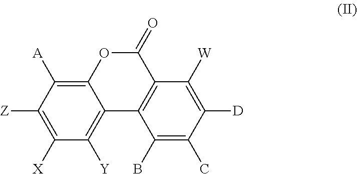

wherein A, B, C, D, W, X, Y and Z are each independently selected from H and OR¹, provided that at least one of A, B, C, D, W, X, Y and Z is OR¹;
each R¹ is independently H or C(═O)R², and at least one R¹ group is C(═O)R²;
each R² is selected from:
CHR³NHR⁴, where R⁴ is H and R³ is a group selected from CH₃, CH₂CH(CH₃)₂, CH(CH₃)CH₂CH₃, CH₂Ph, CH₂-3-(1H-indole), CH₂CH₂SCH₃, CH₂OH, CHOHCH₃, CH₂SH, CH₂SeH and CH₂PhpOH,
wherein said R³ group can optionally be substituted by one or more groups selected from halogen, cyano, nitro, OR^{A} or C₁-C₄ alkyl;
or R³ and R⁴ together with the C and N atoms to which they are attached form a 5-membered heteroalkyl ring,
wherein said heteroalkyl ring can optionally be substituted by one or more groups selected from halogen, cyano, nitro, OR^{A} or C₁-C₃ alkyl,
wherein R^{A} is C₁-C₄ alkyl optionally substituted with one or more halogen, cyano or nitro groups. Compounds of formula (II) are disclosed in WO2015/097231.

In a mode of implementation of the invention, the urolithin is administered as a metabolite of the urolithin, for example a glucuronide or a sulphate. Preferred metabolites are Urolithin A 3-O-glucuronide, Urolithin A 3-O-sulfate, Urolithin B 3-O-glucuronide and Urolithin B 3-O-sulfate.

Those skilled in the art of organic chemistry will appreciate that many organic compounds can form complexes with solvents in which they are reacted or from which they are precipitated or crystallized. These complexes are known as “solvates”. It will be understood by the skilled person that the invention also encompasses solvates of urolithins, as well as solvates of salts, salts, prodrugs, metabolites and derivatives thereof. Solvates include those where the associated solvent is pharmaceutically acceptable. A hydrate (in which the associated solvent is water) is an example of a solvate.

Preferably, the urolithin (or a salt, prodrug, metabolite or derivative thereof) for use in compositions of the invention is micronized. It has been found that micronized urolithin can be dissolved or suspended more rapidly and more effectively than unmicronized urolithin. The micronized urolithin (or a salt, prodrug, metabolite or derivative thereof) preferably has a D₅₀ size of under 100 μm—that is to say that 50% of the urolithin (or a salt, prodrug, metabolite or derivative thereof) by mass has a particle diameter size of under 100 μm. More preferably, the urolithin (or a salt, prodrug, metabolite or derivative thereof) has a D₅₀ size of under 75 μm, for example under 50 μm, for example under 25 μm, for example under 20 μm, for example under 10 μm. More preferably, the urolithin (or a salt, prodrug, metabolite or derivative thereof) has a D₅₀ in the range 0.5-50 μm, for example 0.5 to 20 μm, for example 0.5 μm to 10 μm, for example 1 μm to 10 μm. Preferably, the urolithin (or a salt, prodrug, metabolite or derivative thereof) has a D₉₀ size of under 100 μm—that is to say that 90% of the urolithin (or a salt, prodrug, metabolite or derivative thereof) by mass has a particle diameter size of under 100 μm. More preferably, the urolithin (or a salt, prodrug, metabolite or derivative thereof) has a D₉₀ size of under 75 μm, for example under 50 μm, for example under 25 μm, for example under 20 μm, for example under 15 μm. The urolithin (or a salt, prodrug, metabolite or derivative thereof) preferably has a D₉₀ in the range 5 μm to 100 μm, for example 5 μm to 50 μm, for example 5 to 20 μm. Micronization can be achieved by methods established in the art, for example compressive force milling, hammermilling, universal or pin milling, or jet milling (for example spiral jet milling or fluidised-bed jet milling) may be used. Jet milling is especially suitable.

In some embodiments, the invention is directed to a method for providing beneficial health effects to a subject, the method comprising administration of a combination or composition of the invention to a subject.

In some embodiments, the invention is directed to a combination or composition of the invention for the provision of beneficial health effects to a subject.

In some embodiments, the invention is directed to use of a combination or composition of the invention to provide beneficial health effects to a subject.

In some embodiments, the invention is directed to use of a combination or composition of the invention in the manufacture of a medicament which provides beneficial health effects to a subject.

In some embodiments, the invention is directed to methods of therapy comprising administration of a combination or composition of the invention to a subject.

In some embodiments, the invention is directed to a combination or composition of the invention for use as a medicament.

In some embodiments, the invention is directed to the use of a combination or composition of the invention for the manufacture of a medicament.

In some embodiments, the methods, medicaments or combinations of the invention reduce muscle glycogen and/or protein breakdown during exercise or in aiding muscle recovery after exercise. In some embodiments, the subject is healthy person. In other embodiments, the subject has a muscle wasting condition.

In some embodiments, the methods, medicaments or combinations of the invention decrease muscle wasting in a subject susceptible to muscle wasting. In some embodiments, the subject is healthy person. In other embodiments, the subject has a muscle wasting condition.

In some embodiments, the reduction in protein breakdown during exercise, the muscle recovery after exercise or the degree of muscle wasting can be determined by the loss of leucine, isoleucine and valine in the subject. In some embodiments, the loss of leucine, isoleucine and valine in the subject is reduced by at least 10% after administration of the ketone body ester as compared to the loss of leucine, isoleucine and valine in the subject without administration of the ketone body or ketone body ester.

In some embodiments, the reduction in muscle glycogen breakdown during exercise can be determined by the loss of glycogen in the subject. In some embodiments, the loss of glycogen in the subject is reduced by at least 10% after administration of the ketone body or ketone body ester as compared to the loss of glycogen in the subject without administration of the ketone body or ketone body ester.

Alternatively, the reduction in muscle glycogen and/or protein breakdown during exercise can be determined by the rate of loss of glycogen or protein. In some embodiments, the rate of loss of glycogen or protein is reduced during exercise compared to the level of glycogen without administration of the ketone body or ketone body ester.

In some embodiments, the methods, medicaments or combinations of the invention reduce glycogen and/or protein breakdown in a subject.

In some embodiments, the methods, medicaments or combinations of the invention reduce muscle breakdown or retard muscle wasting in a subject susceptible to muscle breakdown or muscle wasting. In some embodiments, the reduction in muscle breakdown is at least 50% less than that in the same subject under comparable conditions where the methods, medicaments or combinations of the invention have not been used.

In some embodiments, the methods, medicaments or combinations of the invention maintain or improve the muscle power output of a subject. In some embodiments, the methods, medicaments or combinations of the invention maintain or improve skeletal muscle power output. In some embodiments, the methods, medicaments or combinations of the invention maintain or improve power output of other muscle types, for example cardiac muscle.

In some embodiments, the methods, medicaments or combinations of the invention maintain or improve the muscle power output of a subject, wherein the subject is a healthy person.

In some embodiments, the methods, medicaments or combinations of the invention improve the muscle power output of a subject, wherein the improved power output is at least 0.25% relative to a placebo when measured in a controlled test over 30 minutes.

In some embodiments, the methods, medicaments or combinations of the invention improve the muscle power output of a subject, wherein the increased power output is at least 1 Watt relative to a placebo when measured in a controlled test over 30 minutes.

In some embodiments, the methods, medicaments or combinations of the invention find use in improving muscle performance, maintaining or improving muscle function, preventing a decline in muscle function, increasing muscle mass and/or reducing muscle wasting. The improvement in muscle performance, improving or maintaining muscle function, the increase in muscle mass and/or reduction in muscle wasting may be as part of a medical treatment, or it may be for personal preference (“lifestyle”) or cosmetic reasons. Compositions of the invention also find use in reducing muscle inflammation post exercise, and/or enhancing muscle recovery post exercise.

The methods, medicaments or combinations of the invention can be for use as a medicament. The methods, medicaments or combinations can be used as a dietary supplement, as a functional food, functional beverage, or as a medical food.

Methods, medicaments or combinations of the invention are useful in enhancing muscle performance. The invention thus provides methods, medicaments or combinations of the invention for use in enhancing muscle performance. The invention also provides a method of enhancing muscle performance by administering to a subject an effective amount of a composition of the invention. Administration can be self-administration.

The enhanced muscle performance may be one or more improved muscle function, reduced decline in muscle function, improved muscle strength, improved muscle endurance and improved muscle recovery.

The compositions of the invention can thus be used in a method of improving physical endurance (e.g., ability to perform a physical task such as exercise, physical labour, sports activities), inhibiting or retarding physical fatigue, enhancing working capacity, enhancing endurance, and/or reducing muscle fatigue.

Improved muscle function can be particularly beneficial in elderly subjects with reduced muscle function as a result of old age or an age-related condition. For example, a subject who may benefit from improved muscle function may experience a decline in muscle function which then leads to pre-frailty and frailty. Such subjects may not necessarily experience muscle wastage in addition to their decline in muscle function. Some subjects do experience both muscle wasting and a decline in muscle function, for example subjects with sarcopenia. The compositions of the invention may be used in enhancing muscle performance by administering a composition of the invention to a subject who is frail or pre-frail.

In a further embodiment, in the method of enhancing muscle performance, the subject suffers age-related decline in muscle function, age-related sarcopenia, age-related muscle wasting, physical fatigue, muscle fatigue, and/or is frail or pre-frail. In a further embodiment in the method of enhancing muscle performance, the subject suffers physical fatigue or muscle fatigue, and/or wherein the subject is frail or pre-frail.

Muscle performance may be sports performance, which is to say the ability of an athlete's muscles to perform when participating in sports activities. Enhanced sports performance, strength, speed, and endurance are measured by an increase in muscular contraction strength, increase in amplitude of muscle contraction, or shortening of muscle reaction time between stimulation and contraction. Athlete refers to an individual who participates in sports at any level and who seeks to achieve an improved level of strength, speed, or endurance in their performance, such as, for example, body builders, cyclists, long distance runners, and short distance runners. Enhanced sports performance is manifested by the ability to overcome muscle fatigue, ability to maintain activity for longer periods of time, and have a more effective workout.

Methods, medicaments or combinations of the invention may improve physical performance in individuals with a disease, including young and elderly individuals. Compositions of the invention may improve physical performance, for example, short-term performance or long-term performance in healthy individuals, including athletes, non-athletic individuals, sedentary individuals and the elderly. This improvement of performance may be measured by the time spent to walk or run a certain distance (for example, an improved performance during the 6 minute walk test (MWT)), an improved time to run a certain distance, an improved IPAQ score on the international physical activity questionnaire, an increased number of chair-stands in a certain time, or another test designed to measure physical performance.

According to a further embodiment of the invention, there is provided a composition of the invention for use in a method of enhancing physical performance in a subject, for example, in an elite athlete or sub-elite athlete.

In certain embodiments, enhancing physical performance comprises at least one effect selected from the group consisting of enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In further embodiments, enhancing physical performance comprises enhancing muscle performance during a high-intensity aerobic activity. In yet further embodiments, enhancing physical performance comprises increasing aerobic endurance during a high-intensity aerobic activity. In still further embodiments, enhancing physical performance comprises increasing resting metabolic rate (RMR). In certain embodiments, enhancing physical performance results in an improvement in athletic performance. In further embodiments, enhancing physical performance results in an improvement in footrace completion times. In yet further embodiments, enhancing physical performance results in a decrease in Ratings of Perceived Exertion (RPE).

In one embodiment, the enhancing physical performance comprises enhancing muscle performance during a high-intensity aerobic activity.

In one embodiment, the enhancing physical performance comprises increasing aerobic endurance during a high-intensity aerobic activity.

In one embodiment, the enhancing physical performance comprises increasing resting metabolic rate (RMR).

In one embodiment, the enhancing physical performance results in an improvement in athletic performance.

In one embodiment, the enhancing physical performance results in an improvement in footrace completion times.

In one embodiment, the enhancing physical performance results in a decrease in Ratings of Perceived Exertion (RPE).

According to a further embodiment of the invention, there is provided a composition of the invention for use in a method of enhancing physical recovery in a subject, for example, in an elite athlete or sub-elite athlete.

In certain embodiments, physical recovery is enhanced after a high-intensity aerobic activity. In further embodiments, enhancing physical recovery comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In yet further embodiments, enhancing physical recovery comprises enhancing muscle recovery after a high-intensity aerobic activity. In still further embodiments, enhancing physical recovery comprises reducing muscle soreness after a high-intensity aerobic activity. In certain embodiments, enhancing physical recovery comprises lowering creatine kinase (CK) levels in the subject, compared to baseline, following an aerobic activity as measured by area under the plasma concentration-time curve of CK (AUC_{CK}). In further embodiments, enhancing recovery comprises lowering C-reactive protein (CRP) levels in the subject, compared to baseline, following an aerobic activity as measured by area under the plasma concentration-time curve (AUC_{CRP}).

In one embodiment, the enhancement of physical recovery is after high intensity aerobic exercise.

In one embodiment, the enhancing physical recovery comprises enhancing muscle recovery after a high-intensity aerobic activity.

In one embodiment, the enhancing physical recovery comprises reducing muscle soreness after a high-intensity aerobic activity.

In one embodiment, the enhancing physical recovery comprises lowering creatine kinase (CK) levels in the subject, compared to baseline, following an aerobic activity as measured by area under the plasma concentration-time curve of CK (AUC_{CK}).

In one embodiment, the enhancing recovery comprises lowering C-reactive protein (CRP) levels in the subject, compared to baseline, following an aerobic activity as measured by area under the plasma concentration-time curve (AUC_{CRP}).

According to a further embodiment of the invention, there is provided a composition of the invention for use in a method of enhancing endurance in a subject, for example, in an elite athlete or sub-elite athlete.

Compositions of the invention further provide for the improvement of composition. The endurance capacity refers to the time to fatigue when exercising at a constant workload, generally at an intensity <80% VO₂max. Compositions of the invention may improve endurance capacity in individuals with a disease, including young and elderly individuals. Compositions of the invention may improve endurance capacity in healthy individuals, including athletes, non-athletic individuals, sedentary individuals and the elderly. The invention provides for a method of increasing the time to fatigue while performing a specific activity, for example, fitness training, walking, running, swimming, or cycling. This improvement of endurance capacity may be assessed with objective measurements (for example, speed, oxygen consumption or heart rate) or it can be self-reported measurements (for example, using a validated questionnaire).

In certain embodiments, the method comprises enhancing physical endurance during a high-intensity aerobic activity. In further embodiments, enhancing physical endurance comprises at least one effect selected from the group consisting of enhancing muscle recovery, enhancing athletic performance, enhancing running performance, enhancing muscle performance, enhancing aerobic endurance, enhancing the rating of perceived exertion, lowering post exercise fatigue, enhancing muscle recovery, reducing exercise-induced muscle damage, reducing muscle soreness, and enhancing repair of exercise-induced muscle damage. In yet further embodiments, enhancing physical endurance comprises enhancing muscle endurance. In still further embodiments, enhancing physical endurance comprises increasing maximal oxygen consumption (VO₂ₘₐₓ) in the subject.

In one embodiment, the enhancement of physical recovery is during high intensity aerobic exercise.

In one embodiment, the enhancing physical endurance comprises enhancing muscle endurance.

In one embodiment, the enhancing physical endurance comprises increasing maximal oxygen consumption (VO₂max) in the subject.

In certain embodiments, methods of the present disclosure comprise administering to a subject in need thereof an effective amount of a composition of the invention, wherein the subject is an elite athlete or sub-elite athlete. In some such embodiments, the subject is an elite athlete. In certain embodiments, the subject has a VO₂ₘₐₓ of greater than 65 mL kg−1 min−1. In further embodiments, the subject has a 3 km running personal best time below 9 minutes. In other embodiments, the subject is a sub-elite athlete. In certain such embodiments, the subject has a VO₂ₘₐₓ of from about 60 mL kg−1 min−1 to about 65 mL kg−1 min−1. In further embodiments, the subject has a 3 km running personal best time from about 9 minutes to about 10 minutes. In certain embodiments, the age of the subject is from about 18 years to about 45 years.

In some embodiments, the methods, medicaments or combinations of the invention find use in reducing inflammation and/or improving resistance to inflammation.

In some embodiments, the methods, medicaments or combinations of the invention are for the treatment of cognitive dysfunction. In some embodiments, the methods, medicaments or combinations of the invention improve cardiac efficiency. In some embodiments, the methods, medicaments or combinations of the invention improve brain metabolic efficiency.

In some embodiments, the methods, medicaments or combinations of the invention reduce the effects of a neurodegenerative disorder.

In some embodiments, the methods, medicaments or combinations of the invention are for the treatment Alzheimer's disease or Parkinson's disease.

In some embodiments, the methods, medicaments or combinations of the invention are for the treatment muscle impairment or fatigue.

In some embodiments, the methods, medicaments or combinations of the invention maintain or improve cognitive function under fatigue or reduce the adverse effects on cognitive function under fatigue.

### Dosing:

The effective amount of the components of the combination to be taken will vary depending upon the manner of administration, the age, body weight, and general health of the subject. Factors such as the disease state, age, and weight of the subject may be important, and dosage regimens may be adjusted to provide the optimum response.

The daily dose of ketone body (or salt, prodrug, metabolite or derivative thereof) provided by compositions of the invention may be in the range of 20 mg to 5000 mg, for example 20 mg to 4000 mg, for example 200 mg to 4000 mg, for example 20 mg to 3000 mg, for example 20 mg to 2000 mg, for example 100 mg to 1000 mg, for example 100 mg to 800 mg, for example 200 mg to 600 mg, for example 200 mg to 400 mg, for example 200 mg to 300 mg, for example 250 mg. The daily intake of ketone body (or salt, prodrug, metabolite or derivative thereof) may be provided as a single serving, or may be divided between multiple servings.

Alternatively, the daily dose of ketone body (or salt, prodrug, metabolite or derivative thereof) provided by compositions of the invention may be in the range of 2 g to 50 g, for example 5 g to 30 g, for example 10 g to 20 g, for example 2 g, for example 3 g, for example 4 g, for example 5 g, for example 6 g, for example 7 g, for example 8 g, for example 9 g, for example 10 g, for example 11 g, for example 12 g, for example 13 g, for example 14 g, for example 15 g, for example 16 g, for example 17 g, for example 18 g, for example 19 g, for example 20 g, for example 21 g, for example 22 g, for example 23 g, for example 24 g, for example 25 g, for example 26 g, for example 27 g, for example 28 g, for example 29 g, for example 30 g, for example 31 g, for example 32 g, for example 33 g, for example 34 g, for example 35 g, for example 36 g, for example 37 g, for example 38 g, for example 39 g, for example 40 g, for example 41 g, for example 42 g, for example 43 g, for example 44 g, for example 45 g, for example 46 g, for example 47 g, for example 48 g, for example 49 g, for example 50 g. The daily intake of ketone body (or salt, prodrug, metabolite or derivative thereof) may be provided as a single serving, or may be divided between multiple servings.

A unit dose may be in the form of a snack bar; a snack bar of weight 25 g to 150 g, for example 40 g to 100 g may contain the necessary amount of ketone body (or salt, prodrug, metabolite or derivative thereof) (such as 200 mg to 300 mg of ketone body (or salt, prodrug, metabolite or derivative thereof), or another amount mentioned above). A unit dose composition may alternatively be in the form of a drink, for example provided in a container (for example a pouch or a bottle) of a volume suitable for a single dose (for example 50 mL to 500 mL, for example 100 mL to 300 mL). A drink of 100 mL to 300 mL may contain the necessary amount of ketone body (or salt, prodrug, metabolite or derivative thereof). A unit dose composition may alternatively be in the form of a powder to be reconstituted into a drink, for example a suitable quantity of powder for a single dose (for example 5 g to 10 g of powder, containing 200 mg to 300 mg of ketone body (or salt, prodrug, metabolite or derivative thereof)). A reconstituted drink of 100 mL to 500 mL may contain the necessary amount of ketone body (or salt, prodrug, metabolite or derivative thereof). As mentioned below, a composition for use in the invention can take any suitable physical form. It may be in the form of a solid (for example a tablet or a bar), a semi-solid (for example a softgel, capsule (for example a hard capsule) or dragee), a powder or a liquid (including emulsions). The compositions of the invention may be nutritional compositions. The compositions of the invention may be pharmaceutical compositions. The compositions can be in the form of a dietary supplement, as a functional food, functional beverage, or as a medical food or medical nutrition product.

Daily intake of the urolithin (or a salt, prodrug, metabolite or derivative thereof, for example Urolithin A) component is typically in the range of 10 mg to 5 g per day, for example 20 mg to 2500 mg per day, for example 25 mg to 250 mg, for example 25 mg to 500 mg, for example 50 mg to 1500 mg per day, for example 250 mg to 2000 mg, for example 250 mg to 1500 mg per day, for example 50 mg to 1000 mg per day, for example 20 mg to 250 mg per day, for example 250 mg to 1000 mg per day, for example 500 mg to 1000 mg per day, for example 750 mg to 1000 mg per day. In one embodiment, the composition is taken in an amount to provide a dosage of urolithin (or a salt, prodrug, metabolite or derivative thereof) in the range from about 0.2 mg/kg/day to greater than about 100 mg/kg/day. For example, the dosage of urolithin (or a salt, prodrug, metabolite or derivative thereof) may be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 25 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1 mg/kg/day to 25 mg/kg/day, 1 mg/kg/day to 20 mg/kg/day, 1 mg/kg/day to 15 mg/kg/day, 1 mg/kg/day to 10 mg/kg/day, 1 mg/kg/day to 7.5 mg/kg/day, 1 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

A unit dose may be in the form of a snack bar; a snack bar of weight 25 g to 150 g (for example 40 g to 100 g) may contain the necessary amount of urolithin (or a salt, prodrug, metabolite or derivative thereof). A unit dose composition may alternatively be in the form of a drink, for example provided in a container (for example a pouch) of a volume suitable for a single dose (for example 100 mL to 300 mL). A drink of 50 mL to 500 mL (for example 100 mL to 300 mL) may contain the necessary amount of urolithin (or a salt, prodrug, metabolite or derivative thereof). A drink providing the composition of the invention may contain the urolithin (or a salt, prodrug, metabolite or derivative thereof) at a concentration of 0.1 mg per mL to 50 mg per mL, for example 0.5 mg per mL to 10 mg per mL, for example 1 mg per mL to 5 mg per mL. A unit dose may alternatively be in the form of one or more solids such as a compressed tablet. A single compressed tablet may contain a urolithin (or a salt, prodrug, metabolite or derivative thereof) dose of, for example, 25 mg, 50 mg, 75 mg, 100 mg, 150 mg, 200 mg, 250 mg, 300 mg, 350 mg, 400 mg, 450 mg, 500 mg, 600 mg, 700 mg, 800 mg, 900 mg or 1000 mg. A unit dose may alternatively be in the form of one or more semi-solid doses, such as a softgel or paste. A single softgel capsule may contain a urolithin (or a salt, prodrug, metabolite or derivative thereof) dose of, for example, 25 mg, 50 mg, 75 mg, 100 mg, 150 mg, 200 mg, 250 mg, 300 mg, 350 mg, 400 mg, 450 mg or 500 mg, for example 250 mg.

The weight ratio between the ketone body (or a salt, prodrug, metabolite or derivative thereof) and the urolithin (or a salt, prodrug, metabolite or derivative thereof) is generally in the range 500:1 to 1:250; for example 400:1 to 1:250; for example 300:1 to 1:250; for example 300:1 to 1:100; for example 200:1 to 1:100; for example 200:1 to 1:75; for example 200:1 to 1:50; for example 200:1 to 1:25; for example 50:1 to 1:20; for example 25:1 to 1:15; for example 10:1 to 1:10; for example 5:1 to 1:8; for example 5:1 to 1:5; for example 3:1 to 1:5; for example 1:1 to 1:8; for example 1:1 to 1:5; for example 1:2 to 1:6; for example 1:2 to 1:5; for example 1:3 to 1:4. The ratio may also be for example 1:3 to 5:1; for example 1:1 to 8:1; for example 1:1 to 5:1; for example 2:1 to 6:1; for example 2:1 to 5:1; for example 3:1 to 4:1.

A composition of the invention may thus contain 20 mg to 5000 mg of ketone body (or a salt, prodrug, metabolite or derivative thereof) and 10 mg to 5 g of urolithin (or a salt, prodrug, metabolite or derivative thereof); for example 20 mg to 4000 mg of ketone body (or a salt, prodrug, metabolite or derivative thereof) and 10 mg to 5 g of urolithin (or a salt, prodrug, metabolite or derivative thereof); for example 20 mg to 3000 mg of ketone body (or a salt, prodrug, metabolite or derivative thereof) and 10 mg to 5 g of urolithin (or a salt, prodrug, metabolite or derivative thereof); for example 20 mg to 2000 mg of ketone body (or a salt, prodrug, metabolite or derivative thereof) and 10 mg to 5 g of urolithin (or a salt, prodrug, metabolite or derivative thereof); for example 150 mg to 3000 mg of ketone body (or a salt, prodrug, metabolite or derivative thereof) and 10 mg to 5 g of urolithin (or a salt, prodrug, metabolite or derivative thereof); for example 50 mg to 500 mg ketone body (or a salt, prodrug, metabolite or derivative thereof) and 50 mg to 500 mg urolithin (or a salt, prodrug, metabolite or derivative thereof); for example 250 mg to 2000 mg of ketone body (or a salt, prodrug, metabolite or derivative thereof) and 10 mg to 3000 mg of urolithin (or a salt, prodrug, metabolite or derivative thereof); for example 300 mg to 3000 mg of ketone body (or a salt, prodrug, metabolite or derivative thereof) and 10 mg to 3000 mg of urolithin (or a salt, prodrug, metabolite or derivative thereof); for example 200 mg to 600 mg of ketone body (or a salt, prodrug, metabolite or derivative thereof) and 20 mg to 2500 mg of urolithin (or a salt, prodrug, metabolite or derivative thereof); for example 250 mg to 2500 mg of ketone body (or a salt, prodrug, metabolite or derivative thereof) and 100 mg to 2000 mg of urolithin (or a salt, prodrug, metabolite or derivative thereof); for example 200 mg to 400 mg of ketone body (or a salt, prodrug, metabolite or derivative thereof) and 20 mg to 2500 mg of urolithin (or a salt, prodrug, metabolite or derivative thereof); for example 200 mg to 300 mg of ketone body (or a salt, prodrug, metabolite or derivative thereof) and 50 mg to 1000 mg of urolithin (or a salt, prodrug, metabolite or derivative thereof); 200 mg to 300 mg of ketone body (or a salt, prodrug, metabolite or derivative thereof) and 50 mg to 500 mg of urolithin (or a salt, prodrug, metabolite or derivative thereof); 200 mg to 300 mg of ketone body (or a salt, prodrug, metabolite or derivative thereof) and 100 mg to 500 mg of urolithin (or a salt, prodrug, metabolite or derivative thereof). The compositions may further contain, for example, protein, carbohydrates, vitamins and minerals.

Doses of compounds are mentioned herein on a daily dose basis. In many cases, the beneficial effects of the compositions of the invention manifest themselves most when the composition has been taken for an extended period of time, for example 2 weeks or more, for example 4 weeks or more, for example 6 weeks or more, for example 8 weeks, for example 12 weeks or more, for example 16 weeks or more, for example 20 weeks or more, for examples 24 weeks or more.

In the methods of the invention, it is not essential for the urolithin (or salt, prodrug, metabolite or derivative thereof) and the ketone body (or salt, prodrug, metabolite or derivative thereof) to be administered simultaneously as part of a single composition. The urolithin (or salt, prodrug, metabolite or derivative thereof) and the ketone body (or salt, prodrug, metabolite or derivative thereof) can be administered at the same time or separated by a time interval. The urolithin (or salt, prodrug, metabolite or derivative thereof) can be administered first, followed by the ketone body (or salt, prodrug, metabolite or derivative thereof). Alternatively, the ketone body (or salt, prodrug, metabolite or derivative thereof) can be administered first, followed by the urolithin (or salt, prodrug, metabolite or derivative thereof) The invention further provides a kit comprising a urolithin (or salt, prodrug, metabolite or derivative thereof) and ketone body (or salt, prodrug, metabolite or derivative thereof), e.g. for use in the methods of the invention. The urolithin (or salt, prodrug, metabolite or derivative thereof) and ketone body (or salt, prodrug, metabolite or derivative thereof) may be in different physical forms.

Preferably, the urolithin (or salt, prodrug, metabolite or derivative thereof) and the ketone body (or salt, prodrug, metabolite or derivative thereof) are administered simultaneously as part of a single composition. The invention, therefore, also provides a composition comprising a urolithin (or salt, prodrug, metabolite or derivative thereof) and a ketone body (or a salt, prodrug, metabolite or derivative thereof). The composition of the invention can be used in place of the combination of the invention in all of the embodiments described herein. Furthermore, all disclosure relating to the urolithins (or salts, prodrugs, metabolites or derivatives thereof) and ketone bodies (or salts, prodrugs, metabolites or derivatives thereof) and the combination thereof (e.g. dosages) applies to both the combinations of the invention and the compositions of the invention.

In some embodiments, the compositions of the invention comprise a naturally-occurring urolithin (or a salt, prodrug, metabolite or derivative thereof). In some embodiments, the composition of the invention comprises a compound of formula (I) or a salt, prodrug, metabolite or derivative thereof. In some embodiments, the composition of the invention comprises a compound of formula (I) selected from compounds of formula (I) or a salt, prodrug, metabolite or derivative thereof wherein:
Z is OH and W, X and Y are all H;
A, B, W, X and Y are all H, and C, D and Z are all OH (i.e. Urolithin C);
A, B, C, W, X and Y are all H, and D and Z are both OH (i.e. Urolithin A).

In some embodiments, the composition of the invention comprises Urolithin A, Urolithin B, Urolithin C and/or Urolithin D or salts, prodrugs, metabolites or derivatives thereof.

In some embodiments, the composition of the invention comprises Urolithin A or a salt, prodrug, metabolite or derivative thereof.

In some embodiments, the composition of the invention comprises Urolithin A.

In some embodiments, the composition of the invention comprises two or more urolithins (or salts, prodrugs, metabolites or derivatives thereof).

In some embodiments, the compositions of the invention comprise a naturally-occurring ketone body (or a salt, prodrug, metabolite or derivative thereof). In some embodiments, the composition of the invention comprises acetoacetate or a salt, prodrug, metabolite or derivative thereof. In some embodiments, the composition of the invention comprises β-hydroxybutyrate or a salt, prodrug, metabolite or derivative thereof. In some embodiments, the composition of the invention comprises acetone or a salt, prodrug, metabolite or derivative thereof. In some embodiments, the composition of the invention comprises acetoacetate (or a salt, prodrug, metabolite or derivative thereof) and β-hydroxybutyrate (or a salt, prodrug, metabolite or derivative thereof). In some embodiments, the composition of the invention comprises acetoacetate (or a salt, prodrug, metabolite or derivative thereof) and acetone (or a salt, prodrug, metabolite or derivative thereof). In some embodiments, the composition of the invention comprises β-hydroxybutyrate (or a salt, prodrug, metabolite or derivative thereof) and acetone (or a salt, prodrug, metabolite or derivative thereof). In some embodiments, the composition of the invention comprises acetoacetate (or a salt, prodrug, metabolite or derivative thereof), β-hydroxybutyrate (or a salt, prodrug, metabolite or derivative thereof) and acetone (or a salt, prodrug, metabolite or derivative thereof). In some embodiments, the composition of the invention comprises acetoacetate. In some embodiments, the composition of the invention comprises β-hydroxybutyrate. In some embodiments, the composition of the invention comprises acetone. In some embodiments, the composition of the invention comprises acetoacetate and β-hydroxybutyrate. In some embodiments, the composition of the invention comprises acetoacetate and acetone. In some embodiments, the composition of the invention comprises β-hydroxybutyrate and acetone. In some embodiments, the composition of the invention comprises acetoacetate, β-hydroxybutyrate and acetone.

In some embodiments, the composition of the invention comprises more than one ketone body (or salt, prodrug, metabolite or derivative thereof).

### Forms of Compositions:

The compositions of the invention can take any suitable physical form. They may be in the form of a solid (for example a tablet or a bar), a semi-solid (for example a softgel, capsule (for example a hard capsule) or dragee), a powder or a liquid (including emulsions). The compositions of the invention may be nutritional compositions. The compositions of the invention may be pharmaceutical compositions. The compositions can be in the form of a dietary supplement, as a functional food, functional beverage, or as a medical food or medical nutrition product.

Tablet form compositions may be of any suitable type, and they may contain excipients conventional in the art. The excipients can, for example, provide a desired hardness, shelf-life and flavour such that the compostion has an acceptable taste, an attractive appearance and good storage stability. A bar may be of any suitable type and it may contain ingredients conventionally used for the preparation of snack bars.

A snack bar may be a unit dose of the composition of the invention; a snack bar of weight 25 g to 150 g, for example 40 g to 100 g may contain the necessary amount of ketone body (or salt, prodrug, metabolite or derivative thereof) as mentioned above, and the necessary amount of urolithin (or salt, prodrug, metabolite or derivative thereof) as mentioned above.

Semi-solid forms may likewise contain excipients conventional in the art. The excipients can, for example, provide a desired hardness, shelf-life and flavour such that the composition has an acceptable taste, an attractive appearance and good storage stability. Semi-solid forms may be provided for oral administration, or for topical administration.

Powders are commonly used for the supply of nutritional and medical compositions. Powders have the advantage that multiple doses can be provided in a single container, and doses of various sizes can be used from the same supplied container. Powders generally have good storage properties. Powder compositions may also contain excipients conventional in the art. The excipients can, for example, provide a shelf-life, flavour and moisture resistance such that the composition has an acceptable taste, an attractive appearance and good storage stability. The current invention may take the form of a kit comprising a composition comprising a ketone body (or salt, prodrug, metabolite or derivative thereof) together with a separate composition comprising a urolithin (or salt, prodrug, metabolite or derivative thereof), for example ketone body powder composition together with a separate solid or liquid composition containing urolithin (or a salt, prodrug, metabolite or derivative thereof). A solid or liquid composition containing urolithin (or a salt, prodrug, metabolite or derivative thereof), for example a tablet or a drink, or other form described herein, may be provided with instructions for use together with a ketone powder. For example, both ketone body (or a salt, prodrug, metabolite or derivative thereof) and urolithin (or a salt, prodrug, metabolite or derivative thereof) may be in powdered form.

Liquid compositions may be in the form of a medicine, in the form of a drink. Liquid formulations may be solutions, emulsions, slurries or other semi-liquids. Excipients in a liquid composition can, for example, provide a shelf-life, visual appearance, flavour and mouthfeel such that the composition has an acceptable taste, an attractive appearance and good storage stability. Liquid compositions may be provided for oral administration. Liquid compositions may be provided for topical application, for example in the form of creams, ointments or lotions.

A drink may be a unit dose of the composition of the invention, for example provided in a container (for example a pouch or a bottle) of a volume suitable for a single dose (for example 50 mL to 500 mL, for example 100 mL to 300 mL). A drink of 100 mL to 300 mL may contain the necessary amount of ketone body (or salt, prodrug, metabolite or derivative thereof) as mentioned above, and the necessary amount of urolithin (or salt, prodrug, metabolite or derivative thereof) as mentioned above.

Alternatively, a powder to be reconstituted into a drink may be a unit dose of the compositions of the invention, for example a suitable quantity of powder for a single dose (for example 5 g to 10 g of powder, containing 200 mg to 300 mg of ketone body (or salt, prodrug, metabolite or derivative thereof) and). A reconstituted drink of 100 mL to 500 mL may contain the necessary amount of ketone body (or salt, prodrug, metabolite or derivative thereof) as mentioned above, and the necessary amount of urolithin (or salt, prodrug, metabolite or derivative thereof) as mentioned above.

For some uses, compositions of the invention may also be in the form of a solution suitable for injection or intravenous administration.

### Additional Components in Compositions of the Invention

The composition according to the invention may contain additional components beyond the urolithin (or a salt, prodrug, metabolite or derivative thereof) and the ketone body (or a salt, prodrug, metabolite or derivative thereof). The additional components may be compounds that provide health benefits, for example selected from medium chain triglycerides, phospholipids (e.g. phosphatidylcholine), vitamins, minerals, polyunsaturated fatty acids, functional amino acids and other compounds.

The European Pharmacopoeia describes medium-chain triglycerides as the fixed oil extracted from the hard, dried fraction of the endosperm of Cocos nucifera L. (coconut) or from the dried endosperm of Elaeis guineenis Jacq. (African oil palm). The European Pharmacopoeia and the USPNF both have specifications for medium-chain triglycerides that require the presence of particular fatty acids is as follows: caproic acid (C6)≤2.0%; caprylic acid(C8) 50.0-80.0%; capric acid (C10) 20.0-50.0%; lauric acid (C12)≤3.0%; and myristic acid (C14)≤1%.

Medium-chain triglycerides for use in preferred compositions comprise a mixture of triglycerides with fatty acid chains present in the following proportions: C6≤5%; C8 50-70%; C10 30-50%; and C12≤12%, for example C6≤0.5%; C8 55-65%; C10 35-45%; and C12≤1.5%.

Medium-chain triglycerides used in the preferred compositions may be derived from any known or otherwise suitable source.

Amongst vitamins, there may specifically be mentioned Vitamin A, Vitamin C, Vitamin D, Vitamin E, Vitamin B12 and Vitamin K2. As used herein, “vitamin D” refers, to any of known form of vitamin D, and specifically includes vitamin D2 (ergocalciferol), vitamin D3 (cholecalciferol), vitamin D precursors, metabolites and other analogues, and combinations thereof, as well as the various active and inactive forms of vitamin D. For example, vitamin D3 may be provided in its unhydroxylated inactive form as cholecalciferol, or may be provided in its hydroxylated active form as calcitriol.

Creatine has been described as having beneficial effects in the treatment of muscle disorders. It can be included in compositions of the invention. β-Hydroxyl-β-methylbutyrate (HMB) has been described as having beneficial effects in the treatment of muscle disorders. It can be included in compositions of the invention.

Amongst minerals, there may specifically be mentioned calcium salts (for example calcium phosphate), selenium, zinc salt, magnesium salts and iron salts.

Polyunsaturated fatty acids are fatty acids that contain more than one double bond in the backbone. This class includes many important compounds, such as essential fatty acids, e.g., omega-3 and omega-6 fatty acids. Long chain polyunsaturated fatty acids are suitable, and preferably those having at least 20 carbon atoms in the molecule. Such long chain omega-3 fatty acids include cis-11, 14, 17-eicosatrienoic acid (ETE) C20:3, cis-8, 11, 14, 17-eicosatetraenoic acid (ETA) C20:4, cis-5,8, II, 14, 17-eicosapentaenoic acid (EPA) C20:5, cis-7, 10, 13, 16, 19-docosapentaenoic acid (DPA, Clupanodonic acid) C22:5, cis-4, 7, 10, 13, 16, 19-docosahexaenoic acid (DHA) C22:6, cis-9, 12, 15, 18,21-tetracosapentaenoic acid C24:5; cis-6,9,12, 15,18,21-tetracosahexaenoic acid (Nisinic acid) C24:6. Long chain omega-6 fatty acids having at least 20 carbon atoms include cis-11, 14-eicosadienoic acid C20:2, cis-8, 11, 14-eicosatrienoic acid (Dihomo-gamma-linolenic acid) (DGLA) C20:3, cis-5,8,11, 14-eicosatetraenoic acid (Arachidonic acid) (AA) C20:4, cis-13, 16-docosadienoic acid C22:2, cis-7, 10, 13, 16-docosatetraenoic acid (Adrenic acid) C22:4, cis-4, 7, 10, 13, 16-docosapentaenoic acid (Osbond acid) C22:5. The composition according to the invention preferably contains EPA, DHA or a combination of them, for example in an amount from 10 mg to 1000 mg per serving; for example in an amount from 25 mg to 250 mg per serving. For many muscle growth and/or muscle enhancement treatments, it is beneficial for certain particular amino acids to be provided. For example, L-arginine, L-glutamine, lysine and the branched-chain amino acids are considered important. These amino acids are sometimes known as “functional amino acids”. The composition of the invention may include one or more branched-chain amino acids (leucine, isoleucine, and valine). The composition of the invention may include one or both of L-arginine and L-glutamine. The composition of the invention may include lysine.

Pharmaceutical compositions of the invention may include additional pharmaceutically active compounds. For example, a statin may be included. The invention may be provided as a kit comprising a composition of urolithin (or a salt, prodrug, metabolite or derivative thereof) and a ketone body (or a salt, prodrug, metabolite or derivative thereof); and a pharmaceutically active compound, for example a statin.

A composition of the invention may include one or more further agents that are useful for mitochondrial biogenesis or the treatment of mitochondrial disorders. Such compounds include, without limitation, resveratrol, pyrroloquinoline quinone, ubiquinone, sulforaphane, co-enzyme Q10, genistein, hydroxyltyrosol, quercetin, L-carnitine, alpha-lipoic acid, and folinic acid (e.g., as leucovorin).

Additional compounds may further (or alternatively) be included in a composition of the invention, including for example tomatidine, ursolic acid, curcumin, capsaicin, menthol, trolamine salicylate and methylsalicylate.

The additional compound may be an additional active ingredient or supplement, for example a carnitine or a salt thereof. The term carnitine encompasses L-carnitine and derivatives thereof, including acetyl-L-carnitine (ALCAR) and propionyl L-carnitine. Salts of carnitines include tartrate salts, for example in the case of L-Carnitine L-tartrate (LCLT), and glycine salts, e.g. glycine propionyl-L-carnitine (GPLC). When used, carnitines may be administered by any suitable means or dosage form, but commonly carnitines are administered orally, and are dosed daily.

In some exemplary embodiments, the compositions of the present disclosure may comprise, in addition to one or more ketone body (or a salt, prodrug, metabolite or derivative thereof) and urolithin (or a salt, prodrug, metabolite or derivative thereof), one or more additional macronutrients, typically protein, fat or carbohydrate, or two or more of protein, fat and carbohydrate.

Any suitable source of fat or oil of the type commonly used in the preparation of foodstuffs and pharmaceuticals may be used in compositions of the invention. Non-limiting examples of suitable sources of fats for use in the compositions described herein include include coconut oil; fractionated coconut oil; soy oil; corn oil; olive oil; safflower oil; high oleic safflower oil; sunflower oil; high oleic sunflower oil; palm and palm kernel oils; palm olein; canola oil; marine oils; cottonseed oils; polyunsaturated fatty acids such as docosahexaenoic acid (DHA), arachidonic acid (ARA), eicosapentaenoic acid (EPA) and combinations thereof.

Non-limiting examples of suitable carbohydrates or sources thereof for use in the compositions described herein may include maltodextrin, hydrolyzed or modified starch or cornstarch, glucose polymers, corn syrup, corn syrup solids, rice-derived carbohydrates, glucose, fructose, lactose, trehalose, high fructose corn syrup, tapioca dextrin, isomaltulose, sucromalt, maltitol powder, glycerin, fructooligosaccharides, soy fiber, corn fiber, guar gum, konjac flour, polydextrose, honey, sugar alcohols (e.g., maltitol, erythritol, sorbitol), and combinations thereof. Maltodextrin, sucrose and fructose are especially preferred.

Non-limiting examples of suitable proteins or sources thereof for use in the compositions described herein may include hydrolyzed, partially hydrolyzed or non-hydrolyzed proteins or protein sources. They may be derived from any known or otherwise suitable source such as milk (e.g., casein, whey), animal (e.g., meat, fish), cereal (e.g., rice, corn) or vegetable (e.g., soy, pea) sources. Combinations of sources or types of proteins may be used. Non-limiting examples of proteins or sources thereof include intact pea protein, intact pea protein isolates, intact pea protein concentrates, milk protein isolates, milk protein concentrates, casein protein isolates, casein protein concentrates, whey protein concentrates, whey protein isolates, sodium or calcium casemates, whole cow's milk, partially or completely defatted milk, yoghurt, soy protein isolates and soy protein concentrates, and combinations thereof. Combinations of sources or types of proteins may be used. For example, Greek- and Icelandic-style yoghurts are known to commonly have an especially high protein content which makes them especially suitable for use in formulations of the invention. Yoghurts for use in compositions of the invention may contain, for example, from 2 g to 15 g of protein per 100 g. Particularly preferred are yoghurts with a high protein content, for example from 6 g to 15 g per 100 g, for example from 7 g to 15 g per 100 g, for example from 8 g to 15 g per 100 g. Optionally, supplemental protein may also be added to a yoghurt formulation to increase the protein content of the formulation. Yoghurts of the invention may contain live cultures, such as S. thermophilus, L. bulgaricus, L. acidophilus or L. lactis.

The total concentrations or amounts of the protein, fat, carbohydrates and other components vary depending upon the nutritional needs of the intended user.

Additional components in a composition of the invention may be compounds that do not provide health benefits to the subject, but instead improve the composition in some other way, for example its taste, texture or shelf-life as mentioned above. The composition of the invention may thus further contain one or more compounds selected from emulsifiers, colorants, preservatives, gums, setting agents, thickeners, sweeteners and flavourings.

Suitable emulsifiers, colorants, preservatives, gums, setting agents and thickeners are well known in the art of manufacture of emulsions and other semi-liquids. For example preservatives, such as benzoic acid, sorbic acid, phosphoric acid, lactic acid, acetic acid, hydrochloric acid and the soluble salts thereof may be used.

Emulsifiers may include one or more of phosphatidylcholine, lecithin, polysorbates such as polysorbate 60 or polysorbate 80 (Tween-60 and Tween-80), and glycerol monostearate (GMS). Glycerol monostearate is also known as glyceryl monostearate.

Stabilizers may be used in a composition described herein. Many compositions are stable suspensions without the need for an added stabiliser. A stable suspension is one that does not undergo a phase separation over time. For certain compositions, the stability can be improved by inclusion of an added stabiliser. Suitable stabilizers for use in compositions of the invention include glycerol monostearate (GMS), silicon dioxide and vegetable shortening. An exemplary stabiliser is GMS and preferred compositions of the invention contain GMS. Its properties also make GMS a good solvent for phospholipids, such as found in lecithin for example. GMS exists in two polymorphs: the α-form is dispersible and foamy, useful as an emulsifying agent or preservative. The β-form is suitable for wax matrices. The α-form is converted to the β-form when heated at 50° C.

GMS falls into two distinct grades: 40-55 percent monoglycerides, and 90 percent monoglycerides. 40-55 percent monoglycerides as defined by the European Pharmacopoeia describes GMS as a mixture of monoacylglycerols, mostly monostearoylglycerol, together with a quantity of di- and tri-glycerols. In particular, the 40-55 grade contains 40-55% monoacylglycerols, 30-45% diacylglycerols, and 5-15% of triacylglycerols. The 99 percent grade contains not less than 90% of monoglycerides. The monoglycerides in commercial GMS products are mixtures of variable proportions of glyceryl monostearate and glyceryl monopalmitate. The European Pharmacopoeia further divides glyceryl monostearate 40-55 into three types according to the proportion of stearic ester in the mixture. Type 1 contains 40.0-60.0% stearic acid, and the sum of palmitic and stearic acids is 90%. Type 2 contains 60.0-80.0% stearic acid, and the sum of palmitic and stearic acids is 90%. Type 3 contains 90.0-99.0% stearic acid, and the sum of palmitic and stearic acids is 96%. Any form of GMS may be used in the compositions.

In some embodiments, the composition of the invention comprises a urolithin (e.g. Urolithin A) or a salt, prodrug, metabolite or derivative thereof, one or more ketone body or a salt, prodrug, metabolite or derivative thereof, a medium chain triglyceride, and a stabiliser, for example glycerol monostearate. In some embodiments, the composition of the invention comprises a urolithin (e.g. Urolithin A) or a salt, prodrug, metabolite or derivative thereof, one or more ketone body or a salt, prodrug, metabolite or derivative thereof, a medium chain triglyceride, an emulsifier and a stabiliser.

Metal chelators or sequestrants such as sodium calcium salts of ethylenediamine tetra acetic acid (EDTA) may also be used in the compositions of the invention. Other components that may be included in formulations of the invention include polyethylene glycols, silicon dioxide, vegetable shortening and beeswax.

A sweetener may be especially beneficial in a composition of the invention. High potency non-nutritive carbohydrate sweetening agents may be used, for example selected from aspartame, sucrose, potassium acelsufame, saccharin, cyclamates, Stevia, thaumatin and mixtures thereof. Aspartame is especially suitable.

A flavouring may be especially beneficial in a composition of the invention. In a liquid or semi-liquid composition, fruit flavour can be provided by inclusion of a fruit sauce or puree. Typical flavorings include strawberry, raspberry, blueberry, apricot, pomegranate, peach, pineapple, lemon, orange and apple. Generally, fruit flavorings include fruit extract, fruit preserve or fruit puree, with any of a combination of sweeteners, starch, stabilizer, natural and/or artificial flavors, colorings, preservatives, water and citric acid or other suitable acid to control the pH.

For oral preparations, the compositions can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compositions can be formulated into liquid preparations by dissolving, suspending or emulsifying them in an aqueous or nonaqueous solvent, such as vegetable or other similar oils, synthetic aliphatic acid glycerides, esters of higher aliphatic acids or propylene glycol; and if desired with conventional additives such as solubilizers, isotonic agents, suspending agents, emulsifying agents, stabilizers and preservatives. The compositions can be utilized in aerosol formulation to be administered via inhalation. They can be made into suppositories by mixing with a variety of bases such as emulsifying bases or water-soluble bases.

Unit dosage forms for oral administration such as syrups, elixirs, and suspensions may be provided wherein each dosage unit, for example, teaspoonful, tablespoonful, tablet or capsule, contains a predetermined amount of a composition of the invention. Similarly, unit dosage forms for injection or intravenous administration may comprise the compound of the invention in a composition as a solution in sterile water, normal saline or another pharmaceutically acceptable carrier, wherein each dosage unit, for example, mL or L, contains a predetermined amount of a composition of the invention.

TABLE 1
Representative powder composition I:

| Composition | Per 100 g |
|---|---|
| Protein | 10-80 g |
| Carbohydrates | 20-40 g |
| Fat | 0-20 g |
| Polyunsaturated Fatty Acids | 0-5 g |
| Fiber | 0-5 g |
| Vitamins | 0-100% of respective RDAs |
| Minerals | 0-100% of respective RDAs |
| Folic Acid | 0-1000 μg |
| Niacin | 0-100 mg |
| Creatine | 0-20 g |
| Ketone body (e.g. β-hydroxybutyrate) | 0.025-5 g |
| Urolithin A | 0.025-5 g |

TABLE 2
Representative powder composition II:

| Composition | Per 20 g |
|---|---|
| Vitamins | 0-100% of respective RDAs |
| Minerals | 0-100% of respective RDAs |
| Folic Acid | 0-1000 μg |
| Niacin | 0-100 mg |
| Creatine | 0-2 g |
| Excipient filler | 0-4.5 g |
| Ketone body (e.g. β-hydroxybutyrate) | 0.025-5 g |
| Urolithin A | 0.025-5 g |

Bulk powder is generally provided with instructions informing the subject how much of the powder to use for one serving. For example the bulk powder may be supplied in a container accompanied by a scoop of the necessary size to enable the correct amount of powder to be measured out. Powder may be taken neat, mixed with food, or added to water or a juice to make a drink.

TABLE 3
Representative drink composition I:

| Composition | Per 100 mL |
|---|---|
| Protein | 5-15 g |
| (for example 100% Hydrolyzed Whey) | |
| Carbohydrates | 1-20 g |
| Fat | 0-9.5 g |
| Polyunsaturated Fatty Acids | 0-2.5 g |
| Fiber | 0-2 g |
| Vitamins | 0-100% of respective RDAs |
| Minerals | 0-100% of respective RDAs |
| Folic Acid | 0-500 μg |
| Niacin | 0-20 mg |
| Ketone body (e.g. β-hydroxybutyrate) | 0.025-5 g |
| Urolithin A | 10-5000 mg |

TABLE 4
Representative drink composition II:

| Composition | Per 30 mL |
|---|---|
| Vitamins | 0-100% of respective RDAs |
| Minerals | 0-100% of respective RDAs |
| Folic Acid | 0-500 μg |
| Niacin | 0-20 mg |
| Ketone body (e.g. β-hydroxybutyrate) | 0.025-5 g |
| Urolithin A | 10-5000 mg |

TABLE 5
Representative bar composition I:

| Composition | Per bar of 35 g |
|---|---|
| Protein | 6.7 g |
| Carbohydrates | 10-20 g |
| Fat | 0-15 g |
| Polyunsaturated Fatty Acids | 2-6 g |
| Fiber | 0-5 g |
| Niacin | 0-100 mg |
| Vitamins | 0-100% of respective RDAs |
| Minerals | 0-100% of respective RDAs |
| L-Carnitine | 0-500 mg |
| Ketone body (e.g. β-hydroxybutyrate) | 0.025-5 g |
| Urolithin A | 10-5000 mg |

TABLE 6
Representative bar composition II:

| Composition | Per bar of 35 g |
|---|---|
| Vitamins | 0-100% of respective RDAs |
| Minerals | 0-100% of respective RDAs |
| Niacin | 0-100 mg |
| L-Carnitine | 0-500 mg |
| Ketone body (e.g. β-hydroxybutyrate) | 0.025-5 g |
| Urolithin A | 10-5000 mg |

TABLE 7
Representative yoghurt composition

| Composition | Per 100 g Yoghurt |
|---|---|
| Protein | 2-15 g |
| Carbohydrates | 3-20 g |
| Fat | 0-12 g |
| Calcium | 5-20% RDA |
| Fiber | 0-4 g |
| Vitamins | 0-100% of respective RDAs |
| Minerals | 0-100% of respective RDAs |
| Live Cultures | S. thermophilus, L. bulgaricus. L. |
| | acidophilus. L. lactis |
| Ketone body (e.g. β-hydroxybutyrate) | 0.025-5 g |
| Urolithin A | 10-5000 mg |

Representative soft gel capsule formulation (i.e. soft gel capsule containing gelatin shell and fill containing urolithin A)

TABLE 8
Representative fill for soft gel capsule
Fill

| Ingredients | Amount (mg)/Cap | % Total |
|---|---|---|
| Urolithin A | 250 | 7.5 |
| Ketone body (e.g. β-hydroxybutyrate) | 2500 | 75.1 |
| Lecithin NF (35% Total PC) (Epikuron | 284.25 | 8.5 |
| 135 F IP)-E322 | | |
| Medium Chain Triglycerides (MCT) | 284.25 | 8.5 |
| Glycerol Monostearate (40-55) EP, | 11.5 | 0.3 |
| Mono- and Diglycerides NF | | |
| Fill Weight | 3330 | 100 |

TABLE 9
Representative shell for soft gel capsule
Shell

| Ingredients | Amount (mg)/Cap | % Total |
|---|---|---|
| Gelatin EP, NF | 165.97 | 61.5 |
| Glycerol-E422 | 80.1 | 29.7 |
| Water | 21.62 | 8.0 |
| Titanium Dioxide EP-E171 | 1.96 | 0.7 |
| DualDustmaster FD&C Blue #1 | 0.234 | 0.1 |
| (Brilliant Blue FCF-E133) | | |
| Sodium Copper Chlorophyllin Powder | 0.196 | 0.1 |
| (min 95%)-E141 | | |
| Shell Weight | 270 | 100 |
| Total Capsule Weight | 3570 | 100 |

The composition of the invention can be taken as a single treatment or, more commonly, as a series of treatments. In one example, a subject takes a dose before or after exercise. For a subject who is not able to exercise, a dose of the composition may, for example, be taken once, twice or three times per day, or one, two, three, four, five or six times per week. It will also be appreciated that the effective dosage of the compound may increase or decrease over the course of a particular treatment.

### EXAMPLES

The following Examples illustrate the invention.

### Example 1: Effect of the Combination of the Ketone Bodies 3-β-Hydroxybutyrate and Acetoacetate and of Urolithin a on Cellular Energy Production

3-β-hydroxybutyrate and acetoacetate are ketone bodies that are produced by the liver during ketogenesis, and can be used by peripheral tissues to produce ATP. In order to do so, 3-β-hydroxybutyrate and acetoacetate are imported inside mitochondria and converted into acetyl-CoA. Acetyl-CoA can then enter the tricarboxylic acid cycle and be converted into citrate by the citrate synthase. The reduction of NAD⁺ and FADH cofactors will then allow for the production of ATP by oxidative phosphorylation.

C2C12 cells are incubated with 20 μM Urolithin A or vehicle with or without either 3-β-hydroxybutyrate, acetoacetate or a combination of the two for 24 hours to evaluate the effect of 3-β-hydroxybutyrate and acetoacetate alone or in combination with UA on mitochondrial function.

### ATP and Citrate Synthase

Total ATP content and citrate synthase (CS) enzymatic activity are measured as described Mouchiroud, L. et al., Cell 154, 430-441 (2013). Briefly, total ATP content was measured by the CellTiter-Glo® luminescent cell viability assay (Promega), and CS is determined using the CS assay kit (Sigma).

### Respiratory Capacity

Cells are incubated with test compounds in high glucose medium (regular cell culture conditions) and low glucose conditions using successive injections of oligomycin, FCCP (Carbonyl cyanide 4-(trifluoromethoxy)phenylhydrazone) and rotenone/antimycin. This allows the determination of maximal respiratory capacity and uncoupled respiration.

In order to distinguish between anaerobic glycolysis ATP and oxidative phosphorylation ATP, the protocol Seahorse XF ATP is also employed (https://www.agilent.com/en/products/cell-analysis/seahorse-xf-atp).

### Fatty Acid Oxidation

Measurement is performed following the standard protocol for fatty acid oxidation (https://www.agilent.com/cs/pubimages/misc/XF_FAO_Assay_Technical_Brief.pdf). Briefly, once fully differentiated in a 96-well plate, C2C12 myotubes are treated in DMEM-based substrate limited medium, containing 0.5 mM glucose, 1 mM GlutaMAX™ 0.5 mM carnitine, for 24 h. 45 min before the assay. Cells are washed two times with Assay Medium (111 mM NaCl, 4.7 mM KCl, 1.25 mM CaCl₂, 2 mM MgSO₄, 1.2 mM NaH₂PO₄, 2.5 mM glucose, 0.5 mM carnitine, and 5 mM HEPES, adjusted to pH 7.4 at 37° C.) on the day of the assay. Cells are then incubated for 30 min in a non-CO₂ incubator. Just before starting the assay, cells are supplemented with XF Palmitate-BSA FAO Substrate (Seahorse Bioscience) at a final concentration of 166 μM palmitate. Oxygen consumption rate is determined at basal level, and after the addition of FCCP.

### Example 2: Effect of the Combination of the Ketone Bodies 3-s-Hydroxybutyrate and Acetoacetate and of Urolithin a on the Gene Expression of Markers of Mitochondrial Function

C2C12 cells are incubated with 20 μM Urolithin A or vehicle with or without either 3-β-hydroxybutyrate, acetoacetate or a combination of the two for 24 hours to evaluate the effect of 3-β-hydroxybutyrate and acetoacetate alone or in combination with Urolithin A on the expression of genes encoding for mitochondrial proteins.

### Gene Expression Measured by RNA-Seq

Total RNA from C2C12 cells is extracted using TRlzol (Thermo Fisher Scientific) and purified by column using RNeasy® Mini kit (QIAGEN, 74104). Agilent 2100 Bioanalyzer (Agilent RNA 6000 Nano Kit) is used to detect the total RNA samples concentration, RIN, 28S/18S and size. The purity of the samples is tested by NanoDrop™. RNA-seq library preparation is performed as described in D'Amico et al., Mol Cell 2019. RNA-seq run is performed using the Illumina HiSeq 4000 sequencing platform with single reads (1×50 bp) and 30 million reads (+/−3%). The reads obtained are first trimmed out to remove adaptor sequences and then mapped against the human genome (GRCh37). Differential gene expression (DGE) is calculated and genes with a nominal p value <0.05 are selected to run a gene set enrichment analysis using the GSEA tool. The genes sets are taken from the MSIGDB C5 GO collection. Gene sets related to mitochondrial biology with False Discovery Rate (FDR) <0.25 were selected.

### Example 3: Synergistic Anti-Inflammatory Effects of Urolithin a and Beta DL-3-Hydroxybutyric Acid Sodium Salt (BHB)

### Methods

C2C12 Myoblasts, purchased from the American Type Culture Collection (ATCC) (ref. CRL-1772), were seeded at 8000 cells per well in a 96 well plate (Greiner, Kremsmünster, Austria) using a DMEM Glutamax 4.5 g D-glucose (Gibco, ref 31966021) supplemented with foetal bovine serum (FBS) 10% (PanBiotech, ref P190902), 1% PenStrep (Biowest) and 1% HEPES (Biowest). Cells were then differentiated for 6 days using a differentiation medium (DMEM Glutamax 4.5 g D-glucose (Gibco, ref 31966021), 2% Horse serum (Thermofisher, ref 160501229) 1% PenStrep and 1% HEPES). All the media were sterilized with a 0.45 μm filter unit (VWR). After 4 days of the differentiation cells were treated for 24 hours with 1) Urolithin A (UA) resuspended in DMSO, DL-β-Hydroxybutyric acid sodium salt (BHB) (Sigma Reference:H6501-5GA) or a combination of the two at the indicated doses. Cytokine mix (“cytomix”) was added at day 5 in addition of the previous treatment for another 24 hours, cytokines were respectively used at 5 ng (mIFN-γ (Roche ref.11276905001), TNF-α (Peprotech ref. 300-01A) and IL-β (Proteintech ref. 200-01B)). The medium was collected and an ELISA for Interleukin-6 were performed following the instruction provided by the supplier (Proteintech ref. KE10007). Finally, the absorbance at a wavelength of 450 nm was measured using a microplate plate reader (Fluostar Optima, BMG Labtech).

### Results

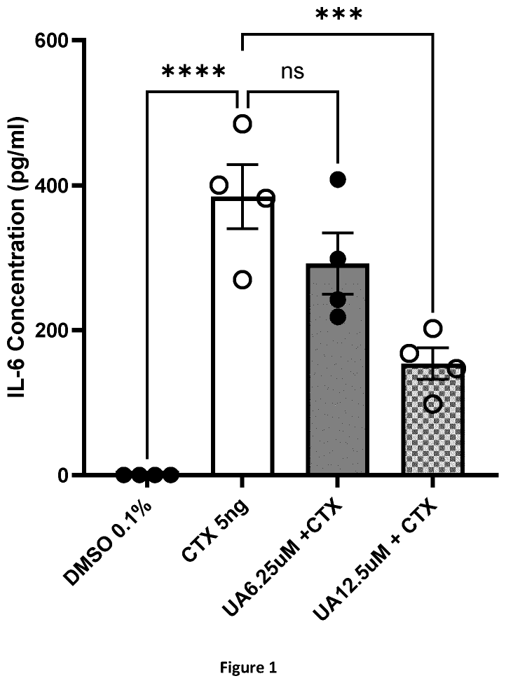

Exposure of C2C12 cells to a mix of pro-inflammatory cytokines (cytomix) led to a significant increase in Interleukin 6 (IL-6) secretion, measured as the concentration of IL-6 in the cell culture media. Urolithin A pre-treatment dose dependently decreased IL-6 secretion, with a dose dependant reduction of 25% at the low 6.25 μM dose and a 60% reduction at the higher 12.5 μM dose (FIG. 1).

To explore potential synergistic effect of Urolithin A (UA) and β-Hydroxybutyric acid (BHB), we treated cells with both compounds, alone or in combination, using a fixed sub-functional UA dose of 6.25 μM and either 5 μM or 25 μM of BHB.

Low dose of BHB (5 μM) alone has no impact on IL-6 secretion (−0.007%). Surprisingly, the combination of low doses of UA (6.25 μM) and low dose of BHB (5 μM) leads to a significant reduction in the inflammatory marker by 56%. This is also statistically significant compared to the reduction of 25% viewed using UA 6.25 μM alone (Table 1).

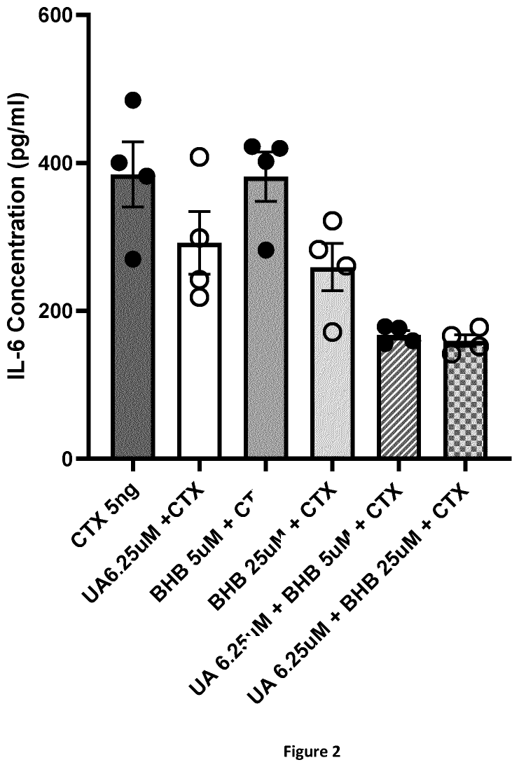

Higher dose of BHB (2 μM) significantly reduces IL-6 secretion by 32%. The combination of low doses of UA (6.25 μM) and a high dose of BHB reduces IL-6 secretion to 58%. This shows the ability of UA at a fixed dose to work synergistically with BHB across a range of doses (FIG. 2 and Table 1).

Ketone bodies such as β-hydroxybutyrate have been shown to reduce inflammation in certain cell-based models. However, this biological effect of BHB was observed only at high concentrations above 2 mM (PMID: 25686106) or 250 μM (PMC6402511). The results of the combination between Urolithin A 6.25 μM and BHB 5 μM indicate a specific and surprising dose window in which UA is able to unlock the potential of low otherwise not-efficient BHB doses.

TABLE 1

| | | Adjusted P |
|---|---|---|
| Tukey's multiple comparisons test | Summary | Value |
| CTX 5 ng vs. UA 6.25 uM +CTX | ns | 0.3438 |
| CTX 5 ng vs. BHB 5 uM + CTX | ns | >0.9999 |
| CTX 5 ng vs. BHB 25 uM + CTX | ns | 0.1022 |
| CTX 5 ng vs. UA 6.25 uM + BHB 5 uM + | ** | 0.0015 |
| CTX | | |
| CTX 5 ng vs. UA 6.25 uM + BHB 25 uM + | ** | 0.001 |
| CTX | | |
| BHB 5 uM + CTX vs. UA 6.25 uM + BHB | ** | 0.0017 |
| 5 uM + CTX | | |
| BHB 25 uM + CTX vs. UA 6.25 uM + BHB | ns | 0.27 |
| 25 uM + CTX | | |

BHB—β-hydroxybutyrate
CTX—cytomix
UA—urolithin A

1. A combination comprising a urolithin (or a salt, prodrug, metabolite or derivative thereof) and a ketone body (or a salt, prodrug, metabolite or derivative thereof).

2. A combination consisting of a urolithin (or a salt, prodrug, metabolite or derivative thereof) and a ketone body (or a salt, prodrug, metabolite or derivative thereof).

3. A composition comprising a urolithin (or a salt, prodrug, metabolite or derivative thereof) and a ketone body (or a salt, prodrug, metabolite or derivative thereof).

4. A combination according to claim 1 or claim 2, or a composition according to claim 3, wherein the urolithin is a compound of formula (I) 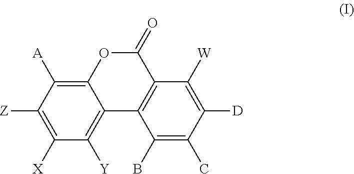
wherein A, B, C, D, W, X, Y and Z are each independently selected from H and OH (or a salt, prodrug, metabolite or derivative thereof).

5. A combination or composition according to claim 4 wherein urolithin is selected from urolithin A, urolithin B, urolithin C and/or urolithin D.

6. A combination or composition according to claim 4 wherein the urolithin is Urolithin A.

7. A combination or composition according to any one of claims 1 to 6 wherein the ketone body is a naturally-occurring ketone body (or a salt, prodrug, metabolite or derivative thereof).

8. A combination or composition according to any one of claims 1 to 7 wherein the ketone body is acetoacetate or a salt, prodrug, metabolite or derivative thereof, and/or β-hydroxybutyrate or a salt, prodrug, metabolite or derivative thereof, and/or acetone or a salt, prodrug, metabolite or derivative thereof.

9. A combination or composition according to any one of claims 1 to 8 wherein the ketone body is acetoacetate and/or β-hydroxybutyrate and/or acetone.

10. A combination or composition according to any one of claims 1 to 9 wherein the ketone body is β-hydroxybutyrate (or a salt, prodrug, metabolite or derivative thereof).

11. A combination or composition according to any one of claims 1 to 10 wherein the ketone body is β-hydroxybutyrate.

12. A method for providing beneficial health effects to a subject, the method comprising administration of a combination or composition according to any one of claims 1 to 11 to a subject.

13. A combination or composition according to any one of claims 1 to 11 for use in the provision of beneficial health effects to a subject.

14. Use of a combination or composition according to any one of claims 1 to 11 to provide beneficial health effects to a subject.

15. Use of a combination or composition according to any one of claims 1 to 11 in the manufacture of a medicament which provides beneficial health effects to a subject.

16. A combination or composition according to any one of claims 1 to 11 for use as a medicament.

17. A combination or composition according to any one of claims 1 to 11 for use for improving muscle performance, maintaining or improving muscle function, preventing a decline in muscle function, increasing muscle mass and/or reducing muscle wasting.

18. A combination or composition according to any one of claims 1 to 11 for use for improving physical endurance, inhibiting or retarding physical fatigue, enhancing working capacity, enhancing endurance, and/or reducing muscle fatigue.

19. A combination or composition according to any one of claims 1 to 11 for use for enhancing physical performance.

20. A combination or composition according to anyone of claims 1 to 11 for use for reducing inflammation and/or improving resistance to inflammation.

21. A kit comprising:
(i) a composition comprising a ketone body or a salt, prodrug, metabolite or derivative thereof; and
(ii) a separate composition comprising a urolithin or a salt, prodrug, metabolite or derivative thereof.

22. A kit according to claim 21 wherein the urolithin or a salt, prodrug, metabolite or derivative thereof is as defined in any one of claims 4 to 6, and/or the ketone body or a salt, prodrug, metabolite or derivative thereof is as defined in any one of claim 7 to 11.

23. A kit according to claim 21 or 22 wherein the urolithin is urolithin A.

24. A kit according to any one of claims 21 to 23 wherein the ketone body is selected from acetoacetate and/or β-hydroxybutyrate and/or acetone.
